(19) United States
(12) Patent Application Publication
(10) Pub. No.: US 2021/0402004 A1
(43) Pub. Date: Dec. 30, 2021

(54) NANOPARTICLE STRUCTURE AND METHOD OF FORMING THE SAME

(71) Applicants: Seoul National University R&DB Foundation, Seoul (KR); INSTITUTE FOR BASIC SCIENCE, Daejeon (KR)

(72) Inventors: Taeghwan HYEON, Seoul (KR); Sungjoong LEE, Seoul (KR); Min SOH, Seoul (KR); Boomin CHOI, Seoul (KR)

(21) Appl. No.: 17/291,246

(22) PCT Filed: Nov. 5, 2019
(86) PCT No.: PCT/KR2019/014879

§ 371 Date: May 4, 2021

(30) Foreign Application Priority Data
Nov. 6, 2018 (KR) 10-2018-0134987

(51) Int. Cl. A61K 47/69

(52) U.S. Cl. CPC A61K 47/6923; B82Y 5/00; A61K 47/6929

(57) ABSTRACT

A nanoparticle structure and method of forming the same are provided. The nanoparticle structure comprises a metal oxide nanoparticle. The metal oxide nanoparticle may comprise ceria and zirconia. The nanoparticle structure may further comprise an antibody connected to the metal oxide nanoparticle. The method comprises forming a metal oxide nanoparticle. The method may further comprise connecting an antibody to the metal oxide nanoparticle.

### TECHNICAL FIELD

The present invention relates to a nanoparticle structure and method of forming the same.

### BACKGROUND ART

Neuropathic pain means an abnormality in the physiological phenomena of the central nervous system. Pain perception even in the absence of stimuli (spontaneous pain), pain perception against non-noxious stimuli (allodynia), and exaggerated pain perception against noxious stimuli (hyperalgesia) is represented by a reduced stimulation threshold. Microglia, immune cells residing in the central nervous system, are one of the major factors involved in the pathogenesis of neuropathic pain.

Impaired neural induction signals induce spinal cord microglia activation and subsequent pain related gene expression such as IL-1β, IL-6 and TNF-α. This sensitizes neurons or neural circuits that carry pain to trigger pain-centered sensitization.

In addition, spinal nerve transection (SNT) induces the generation of reactive oxygen species in the microglia of the spinal cord to cause neuropathic pain. The generation of the reactive oxygen species derived from NADPH oxidase 2 (Nox2) plays an important role in neuronal injury-induced spinal microglia activation and subsequent pain hypersensitivity.

### DISCLOSURE

### Technical Problem

In order to solve the above mentioned problems, the present invention provides a novel nanoparticle structure.

The present invention provides a nanoparticle structure having a good therapeutic effect.

The other objects of the present invention will be clearly understood by reference to the following detailed description and the accompanying drawings.

### Technical Solution

A nanoparticle structure according to the embodiments of the present invention comprises a metal oxide nanoparticle.

The metal oxide nanoparticle may comprise ceria. The metal oxide nanoparticle may further comprise zirconia. The metal oxide nanoparticle may have a formula of Ce_{0.7}Zr_{0.3}O₂.

The nanoparticle structure may further comprise an antibody connected to the metal oxide nanoparticle. The antibody may comprise a microglia targeting antibody. The nanoparticle structure may relieve neuropathic pain. The nanoparticle structure may inhibit microglia activation.

A method of forming a nanoparticle structure according to the embodiments of the present invention comprises forming a metal oxide nanoparticle.

The metal oxide nanoparticle may be formed by putting cerium(III) acetylacetonate hydrate and zirconium(IV) acetylacetonate hydrate in oleylamine and heating the cerium(III) acetylacetonate hydrate and the zirconium(IV) acetylacetonate hydrate to react.

The method may further comprise connecting an antibody to the metal oxide nanoparticle.

Connecting the antibody to the metal oxide nanoparticle may comprise connecting phospholipid-PEG to the metal oxide nanoparticle and reacting the antibody with the phospholipid-PEG.

The antibody may react with NHS-ester before the antibody is connected to the metal oxide nanoparticle and may react with the phospholipid-PEG to be connected to the metal oxide nanoparticle by amide bond.

### Advantageous Effects

According to embodiments of the present invention, a novel nanoparticle structure having a good therapeutic effect may be implemented. For example, the nanoparticle structure may inhibit microglia activation. The targeted delivery of the nanoparticle structure induces improved elimination of inflammatory cytokines and reactive oxygen species in microglia to enable down-regulation of hyperactivated microglia. Thereby, neuropathic pain induced by microglia activation can be suppressed. In addition, the nanoparticle structure may be used for the treatment of various diseases related to microglia activation and may be applied to other targeted treatments other than microglia.

### DESCRIPTION OF DRAWINGS

FIG. 1 schematically shows a nanoparticle structure and method of forming the same according to an embodiment of the present invention.

FIG. 2 shows a TEM image of a nanoparticle structure according to an embodiment of the present invention.

FIG. 3 shows a graph of XRD analysis of a nanoparticle structure according to an embodiment of the present invention.

FIG. 4 shows a graph of XPS analysis of cerium.

FIG. 5 shows a graph of XPS analysis of zirconium.

FIG. 6 shows a graph of FCS analysis of a nanoparticle structure according to an embodiment of the present invention.

FIG. 7 shows the hydrodynamic diameter and potential of a nanoparticle structure according to an embodiment of the present invention.

FIG. 8 shows the reactive oxygen species scavenging performance of a nanoparticle structure according to an embodiment of the present invention.

FIG. 9 shows an image of microglia cells treated with nanoparticle structures according to an embodiment of the present invention.

FIG. 10 is a view for explaining the microglia absorption efficiency of nanoparticle structures according to an embodiment of the present invention.

FIG. 11 shows the expression level of pain-mediating gene after treatment of nanoparticle structures according to an embodiment of the present invention.

FIGS. 12 to 16 are views for explaining microglia specific delivery of nanoparticle structures according to an embodiment of the present invention.

FIGS. 17 to 21 are views for explaining an analgesic effect of nanoparticle structures according to an embodiment of the present invention.

### BEST MODE

Hereinafter, a detailed description will be given of the present invention with reference to the following embodiments. The purposes, features, and advantages of the present invention will be easily understood through the following embodiments. The present invention is not limited to such embodiments, but may be modified in other forms. The embodiments to be described below are nothing but the ones provided to bring the disclosure of the present invention to perfection and assist those skilled in the art to completely understand the present invention. Therefore, the following embodiments are not to be construed as limiting the present invention.

The size of the element or the relative sizes between elements in the drawings may be shown to be exaggerated for more clear understanding of the present invention. In addition, the shape of the elements shown in the drawings may be somewhat changed by variation of the manufacturing process or the like. Accordingly, the embodiments disclosed herein are not to be limited to the shapes shown in the drawings unless otherwise stated, and it is to be understood to comprise a certain amount of variation.

A nanoparticle structure according to the embodiments of the present invention comprises a metal oxide nanoparticle.

The metal oxide nanoparticle may comprise ceria. The metal oxide nanoparticle may further comprise zirconia. The metal oxide nanoparticle may have a formula of Ce_{0.7}Zr_{0.3}O₂.

The nanoparticle structure may further comprise an antibody connected to the metal oxide nanoparticle. The antibody may comprise a microglia targeting antibody. The nanoparticle structure may relieve neuropathic pain. The nanoparticle structure may inhibit microglia activation.

A method of forming a nanoparticle structure according to the embodiments of the present invention comprises forming a metal oxide nanoparticle.

The metal oxide nanoparticle may be formed by putting cerium(III) acetylacetonate hydrate and zirconium(IV) acetylacetonate hydrate in oleylamine and heating the cerium(III) acetylacetonate hydrate and the zirconium(IV) acetylacetonate hydrate to react.

The method may further comprise connecting an antibody to the metal oxide nanoparticle.

Connecting the antibody to the metal oxide nanoparticle may comprise connecting phospholipid-PEG to the metal oxide nanoparticle and reacting the antibody with the phospholipid-PEG.

The antibody may react with NHS-ester before the antibody is connected to the metal oxide nanoparticle and may react with the phospholipid-PEG to be connected to the metal oxide nanoparticle by amide bond.

### Embodiments

[Synthesis of Ce_{0.7}Zr_{0.3}O₂ Nanoparticles]

0.36 mg of cerium(III) acetylacetonate hydrate and 0.14 mg of zirconium(IV) acetylacetonate hydrate are added to 15 ml of oleylamine. The mixture is sonicated at 20° C. for 10 minutes and heated to 80° C. at a heating rate of 2° C./min. The mixture was reacted at 80° C. for 1 day and cooled to 20° C. The product was washed with acetone (100 ml) and collected by carrying out centrifugation at 5000 rpm several times. The resulting Ce_{0.7}Zr_{0.3}O₂ nanoparticles (7CZ nanoparticles) are dispersed in chloroform at a final concentration of 10 mg/ml. The 7CZ nanoparticle may have a size of about 2 nm.

[Phospholipid-PEG-FITC Bonding]

6 mg of DSPE-PEG(2000)-amine and 0.6 mg of fluorescein isothiocyanate (FITC) are dispersed in 0.6 ml of chloroform. The mixture is kept heated while stirred at 40° C. for 4 hours. After 4 hour reaction, FITC is covalently bonded to the amine group in the PEG.

[CD11b Antibody Bonding to NHS-Ester]

In the EDC coupling reaction, 1.5 mg of CD11b or FITC-CD11b antibody is added to a mixed solution of 30 mg of EDCI, 18 mg of NHS(N-Hydroxysuccinimide)-ester, 60 μl of TEA, and 9 ml of deionized water. The mixture is shaken for 2 hours at room temperature. After the reaction for 2 hours, the carboxylic acid group of the CD11b antibody is covalently bonded to the NHS-ester.

[Synthesis of Phospholipid-PEG Capped 7CZ Nanoparticles, 7CZ-FITC Nanoparticles]

To form water-dispersible 7CZ nanoparticles, 1.5 ml of 7CZ nanoparticles are dispersed in chloroform (10 mg/ml), and mixed with 4.5 ml mixture of PEG(2000) in the chloroform (10 mg/ml, mPEG(200)-PE:DSPE-PEG(2000)-amine=2:1). In the case of 7CZ-FITC nanoparticles, the same amount of 7CZ nanoparticles dispersed in chloroform was mixed with 4.5 ml mixture of PEG(2000) and PEG(2000)-FITC in the chloroform (10 mg/ml, mPEG(200)-PE:DSPE-PEG(2000)-amine:DSPE-PEG(2000)-FITC=10:3:2). Each sample is treated on a rotary evaporator and treated in a vacuum oven at 70° C. for 2 hours and the chloroform is completely removed. The resulting product is dispersed in 5 ml of deionized water to form a transparent colloidal suspension. Phospholipid-PEG residues are removed by carrying out filtering using a 0.4 μm filter and ultracentrifugation several times. The clarified product of each sample is maintained in deionized water.

[CD11b Antibody Bonding to 7CZ Nanoparticles or 7CZ-FITC Nanoparticles]

To bond CD11b or FITC-CD11b antibodies to 7CZ nanoparticles or 7CZ-FITC nanoparticles, a water-dispersed sample (7CZ nanoparticles or 7CZ-FITC nanoparticles) was added to the intermediate of the antibody-NHS ester mixture formed in deionized water. This mixture comprising nanoparticles and antibodies is stirred at room temperature for 12 hours. The reaction product is washed several times after ultracentrifugation. Purified 7CZ-Ab nanoparticles (antibody-bonded 7CZ nanoparticles) or 7CZ-Ab-FITC nanoparticles are finally dispersed in deionized water.

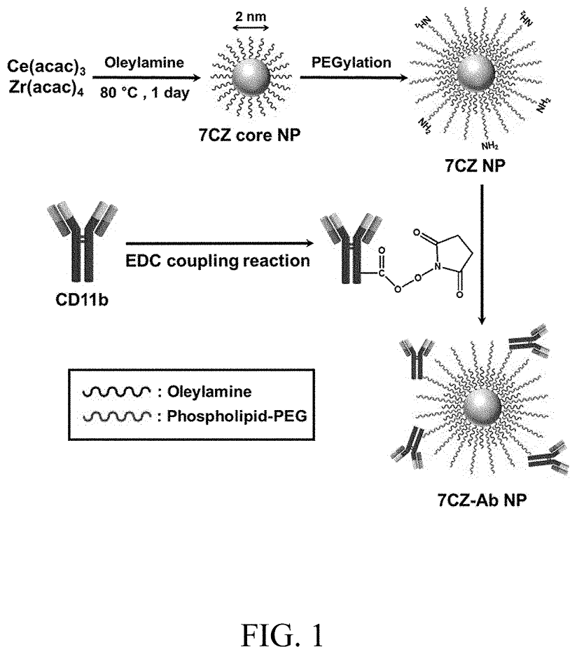

FIG. 1 schematically shows a nanoparticle structure and method of forming the same according to an embodiment of the present invention.

Referring to FIG. 1, cerium(III) acetylacetonate hydrate and zirconium(IV) acetylacetonate hydrate are added to oleylamine. The mixture is sonicated at 20° C. for 10 minutes and heated to 80° C. at a heating rate of 2° C./min. The mixture was reacted at 80° C. for 1 day and cooled to 20° C. As a result, 7CZ nanoparticles are formed. The 7CZ nanoparticles include ceria and zirconia. The 7CZ nanoparticles may have a size of about 2 nm.

The 7CZ nanoparticles are PEGylated. The 7CZ nanoparticles may have water dispersibility by the PEGylation. The PEGylation may be performed by reacting the 7CZ nanoparticles with phospholipid-PEGs.

The CD11b antibody is bonded to the NHS-ester through the EDC coupling reaction. The carboxylic acid group of the CD11b antibody is covalently bonded to the NHS-ester by the EDC coupling reaction.

The water-dispersed 7CZ nanoparticles are added to deionized water in which the antibody-NHS ester is formed, and the CD11b antibodies are bonded to the 7CZ nanoparticles to form antibody-bonded 7CZ nanoparticles (7CZ-Ab nanoparticles). Bonding of the CD11b antibody can be achieved by a stable amide bond between the antibody and the phospholipid-PEG shell.

As described above, ceria-zirconia nanoparticles (Ce_{0.7}Zr_{0.3}O₂; 7CZ nanoparticles) functionalized with antibodies can be synthesized through a non-hydrous sol gel reaction.

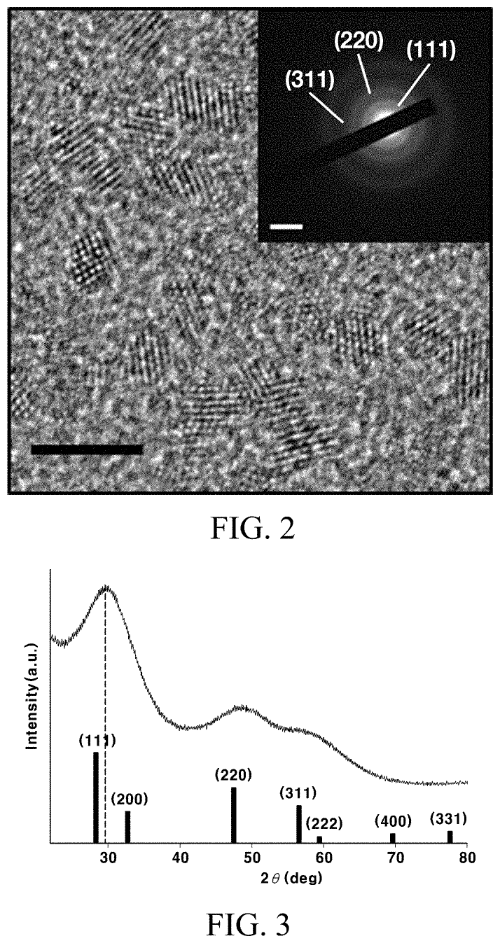

FIG. 2 shows a TEM image of a nanoparticle structure according to an embodiment of the present invention, and FIG. 3 shows a graph of XRD analysis of a nanoparticle structure according to an embodiment of the present invention.

Referring to FIGS. 2 and 3, HRTEM (high-resolution transmission electron microscopy) images show discrete and well-defined lattice patterns of uniformly sized core 7CZ nanoparticles of about 2 nm. In addition, SAED (selected area electron diffraction) and XRD (X-ray diffraction) data show the fluorite-based cubic structure of 7CZ nanoparticles.

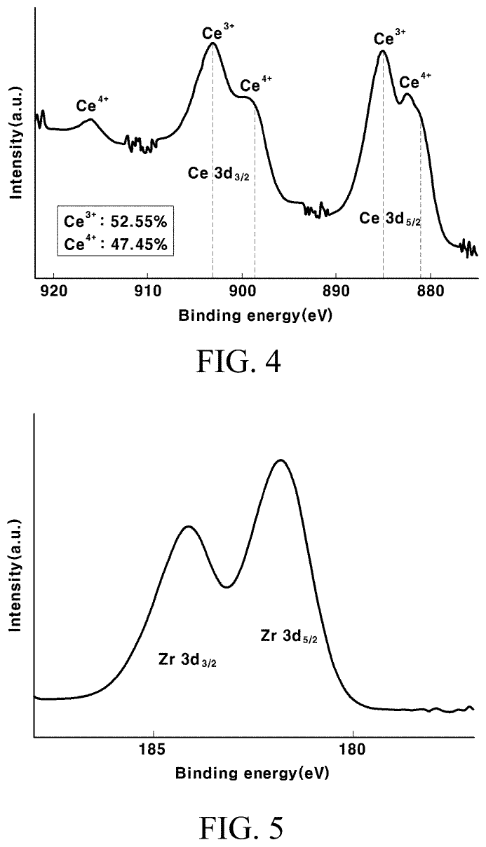

FIG. 4 shows a graph of XPS analysis of cerium and FIG. 5 shows a graph of XPS analysis of zirconium.

Referring to FIGS. 4 and 5, since tetragonality increases as Zr⁴⁺ ions are inserted into the ceria nanoparticles, a slight peak shift of the 7CZ nanoparticles is observed in the XRD pattern. Since the above synthesis method enables 7CZ nanoparticles to form a solid solution, a high Ce³⁺/Ce⁴⁺ ratio (Ce³⁺ ion ratio in 7CZ nanoparticles: 52.55%) in 7CZ nanoparticles is confirmed by XPS (X-ray photoelectron spectroscopy) analysis.

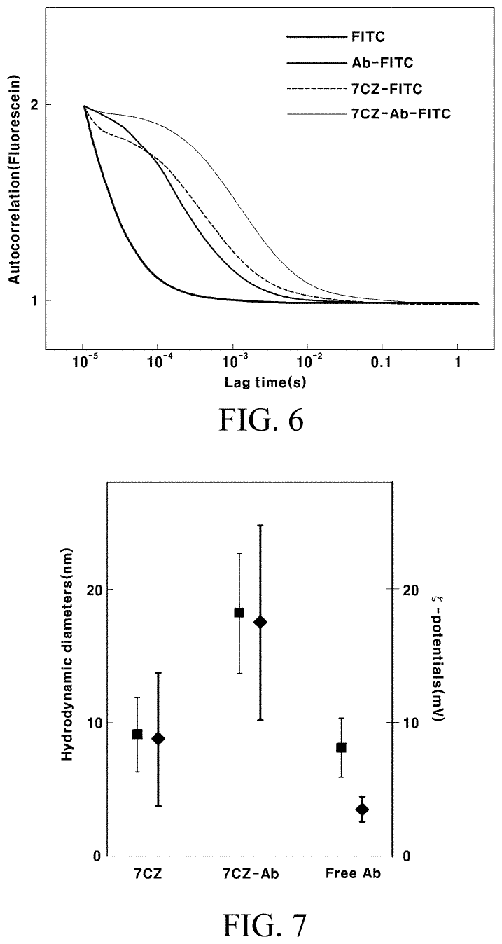

FIG. 6 shows a graph of FCS analysis of a nanoparticle structure according to an embodiment of the present invention.

Referring to FIG. 6, in FCS (fluorescence correlation spectroscopy) analysis after PEGylation and functionalization (antibody bonding) of nanoparticles, 7CZ-Ab nanoparticles exhibit increased diffusion time and decreased diffusion coefficient than free antibody and 7CZ nanoparticles.

FIG. 7 shows the hydrodynamic diameter and potential of a nanoparticle structure according to an embodiment of the present invention.

Referring to FIG. 7, after PEGylation and functionalization (antibody bonding) of nanoparticles and in DLS (dynamic light scattering) analysis, the hydrodynamic diameter (HD) and ζ potential of the 7CZ nanoparticles are 9.1 nm and −8.8 mV, but the hydrodynamic diameter and potential of 7CZ-Ab nanoparticles are 18.2 nm and −17.5 mV. Compared to free antibodies and 7CZ nanoparticles, the increased diffusion time and hydrodynamic diameter and the decreased diffusion coefficient and potential of 7CZ-Ab nanoparticles are due to the successful bonding of the antibody to the nanoparticles.

In order to compare the antibody effect on the reactive oxygen species scavenging activity of nanoparticles, SOD (superoxide dismutase) mimetic activity, CAT (catalase) mimetic activity, and HORAC (hydroxyl radical protection factor) activity were analyzed.

Superoxide anion scavenging activity was evaluated using the SOD assay kit (Sigma-Aldrich). 20 μl of each sample with a final concentration of 0.1 mM was added to 160 μl of WST-1(2-(4-iodophenyl)-3-(4-nitrophenyl)-5-(2,4-disulfophenyl)-2H-tetrazolium sodium salt) solution. Then, 20 μl of a xanthine oxidase solution as a superoxide anion generator was added to each microplate well. After maintaining the constant temperature at 37° C. for 20 minutes, the absorbance of each well was measured at 450 nm using a multiple plate reader. Since the absorbance is proportional to the amount of superoxide anion, the inhibition rate of superoxide was calculated by quantifying the reduction in color development.

CAT mimetic activity was measured using a CAT assay kit (Amplex Red hydrogen peroxide/peroxidase assay kit, Molecular Probes, Inc.). 10 μl of each sample with a final concentration of 0.1 mM was mixed with 40 μl of a final concentration of 5 μM H₂O₂ solution in each microplate well. After incubating for 20 minutes, Amplex Red reagent/HRP solution was added to each well, and light was blocked for each sample, and the temperature was maintained at 25° C. for 30 minutes. Fluorescence was measured using a multiple plate reader.

The hydroxyl radical scavenging activity was evaluated using a HORAC assay kit (Cell Biolabs, Inc.). 20 μl of each sample with a final concentration of 0.1 mM was added to 140 μl of a fluorescent probe. After incubating at 25° C. for 30 minutes, 20 μl of a hydroxyl initiator and 20 μl of Fenton reagent were added to each microplate well to generate hydroxyl radicals. After shaking for 15 seconds and maintaining the constant temperature at 25° C. for 20 minutes, fluorescence was measured using a multiple plate reader.

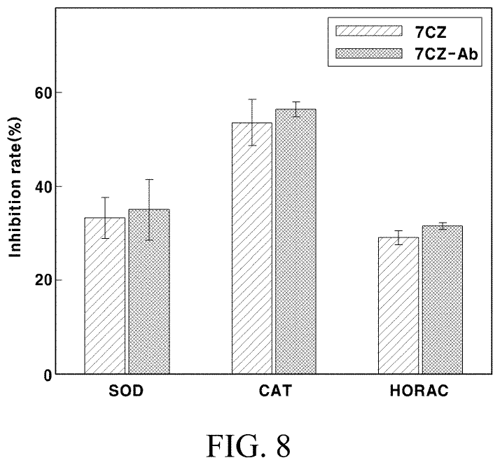

FIG. 8 shows the reactive oxygen species scavenging performance of a nanoparticle structure according to an embodiment of the present invention.

Referring to FIG. 8, in the performed SOD mimetic activity assay, CAT mimetic activity assay, and HORAC activity assay, antibody bonding to nanoparticles shows the same performance as that performed by 7CZ nanoparticles. The antibody bonding does not make a difference for catalytic activity to remove reactive oxygen species. These results indicate that the removal of reactive oxygen species in the aqueous medium is not related to the antibody bonding, but related to the ability of metal oxide nanoparticles (core nanoparticles).

To compare and test the microglia uptake of 7CZ nanoparticles and 7CZ-Ab nanoparticles, two concentrations of FITC-bonded nanoparticles, 0.01 mM and 0.02 mM were treated in pure microglia culture for 3 hours and 15 hours. Afterwards, the FITC signal in the cell density was analyzed using ICC and FACS.

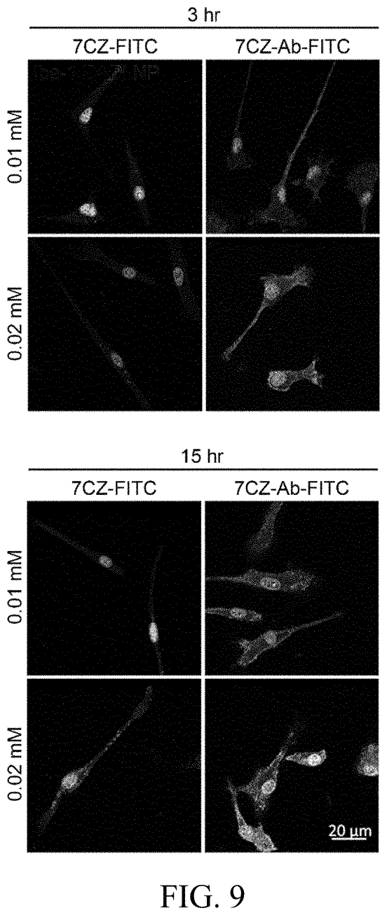

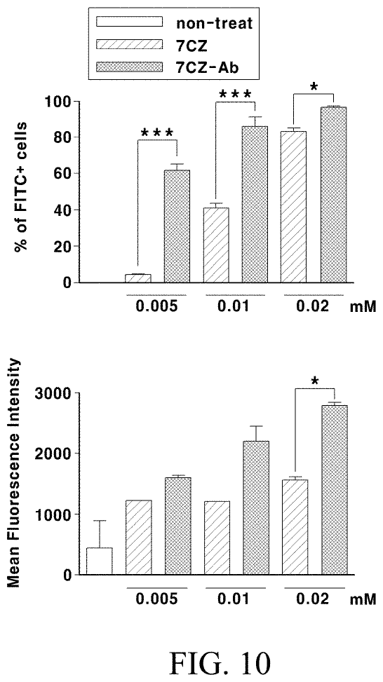

FIG. 9 shows an image of microglia cells treated with nanoparticle structures according to an embodiment of the present invention and FIG. 10 is a view for explaining the microglia absorption efficiency of nanoparticle structures according to an embodiment of the present invention.

Referring to FIG. 9, a higher fluorescence signal was observed in the treatment of 7CZ-Ab nanoparticles, and the 7CZ-Ab nanoparticles exhibited greater microglia absorption efficiency than the 7CZ nanoparticles.

Referring to FIGS. 9 and 10, 7CZ nanoparticles were not internalized in microglia at the time point of 3 hours, but 7CZ-Ab nanoparticles were absorbed into microglia cells. This indicates that 7CZ-Ab nanoparticles are internalized in microglia at a faster rate than 7CZ nanoparticles. Therefore, FACS analysis showed that the uptake of 7CZ-Ab nanoparticles by microglia cells was significantly increased than that of 7CZ nanoparticles. After treatment with nanoparticles at a low concentration (0.005 mM) for 3 hours, the percentage of microglia cells positive for 7CZ-Ab-FITC was about 60% and the percentage of microglia cells positive for 7CZ-FITC was less than 5%. At a concentration of 0.01 mM, 7CZ-Ab nanoparticles were absorbed by more than 80% of microglia cells compared to 7CZ nanoparticles uptake by about 40% of microglia cells.

Although the number of FITC-positive microglia cells was similar at higher concentrations of nanoparticle-treated cells (0.02 mM), the MFI of 7CZ-Ab nanoparticle-treated cells was much higher than that of 7CZ nanoparticle-treated cells. This indicates that uptake of 7CZ-Ab nanoparticles by glia at higher concentration is higher. These data show that CD11-Ab bonding to the 7CZ nanoparticle increases its targeting capacity for microglia and absorption efficiency.

Pro-inflammatory mediators such as IL-1β, IL-6, and NO, which are induced in activated spinal cord microglia, contribute to the development of neuropathic pain. In addition, reactive oxygen species are involved in the induction of pro-inflammatory gene expression. Therefore, it was tested whether 7CZ nanoparticles and 7CZ-Ab nanoparticles inhibit pain-mediating gene expression in glia cells in vitro.

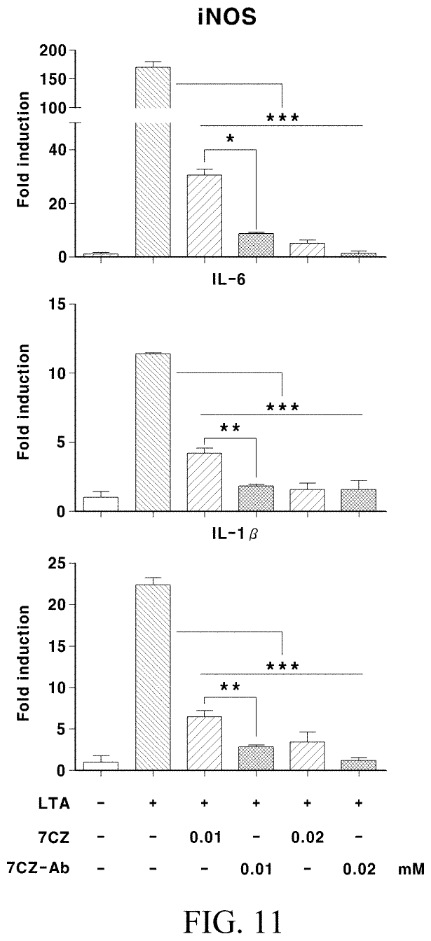

FIG. 11 shows the expression level of pain-mediating gene after treatment of nanoparticle structures according to an embodiment of the present invention.

Referring to FIG. 11, the mixed glia cells were treated with LTA (1 μg/ml) or with LTA with 7CZ nanoparticles or 7CZ-Ab nanoparticles of 0.01 mM, 0.02 mM, and 0.04 mM for 15 hours. In the LTA treatment, mRNA expressions of iNOS, IL-6 and IL-1β increased by 169, 12 and 22 times, respectively, but treatment with 7CZ or 7CZ-Ab nanoparticles significantly decreased according to the dose.

Treatment with 7CZ-Ab nanoparticles has a higher inhibitory effect than treatment with 7CZ nanoparticles as indicated by the remarkably high reduction rate of cytokines and iNOS by treatment with 7CZ-Ab nanoparticles. LTA-induced mRNA expression of iNOS, IL-6 and IL-1β decreased by 95%, 86% and 91%, respectively, with 0.1 mM 7CZ-Ab nanoparticle treatment and decreased by 82%, 63%, and 71% with 0.1 mM 7CZ nanoparticle treatment. This indicates that 7CZ-Ab nanoparticles better inhibit pain-mediating gene expression in microglia than 7CZ nanoparticles.

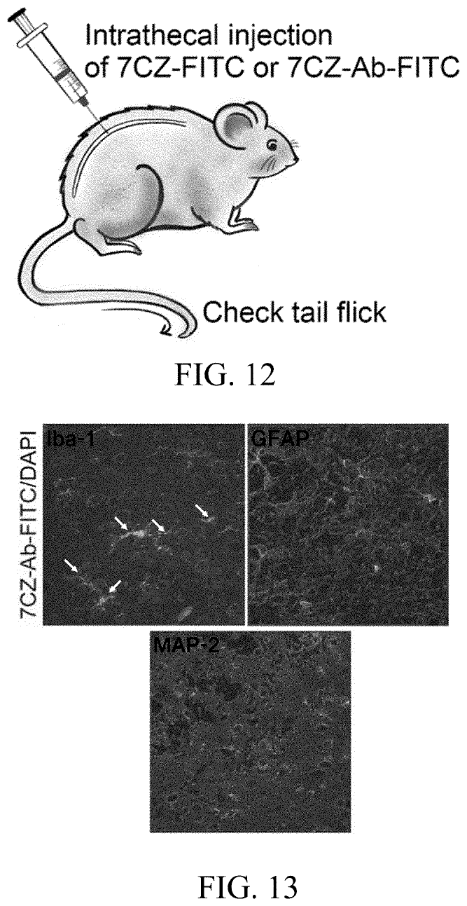

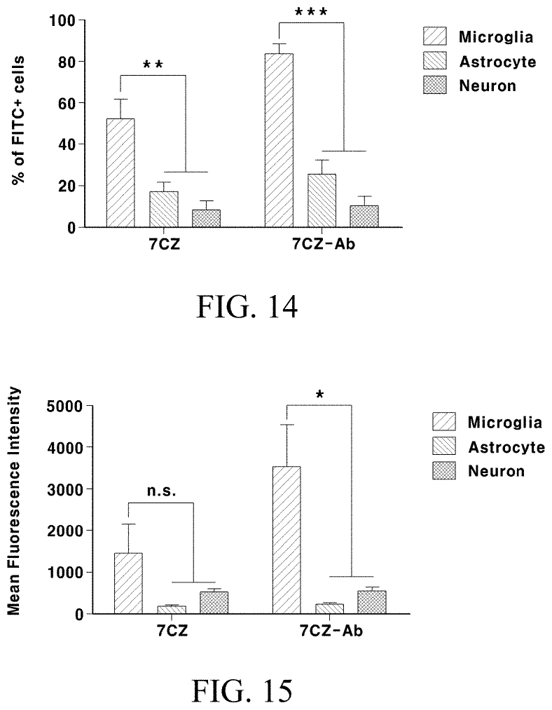

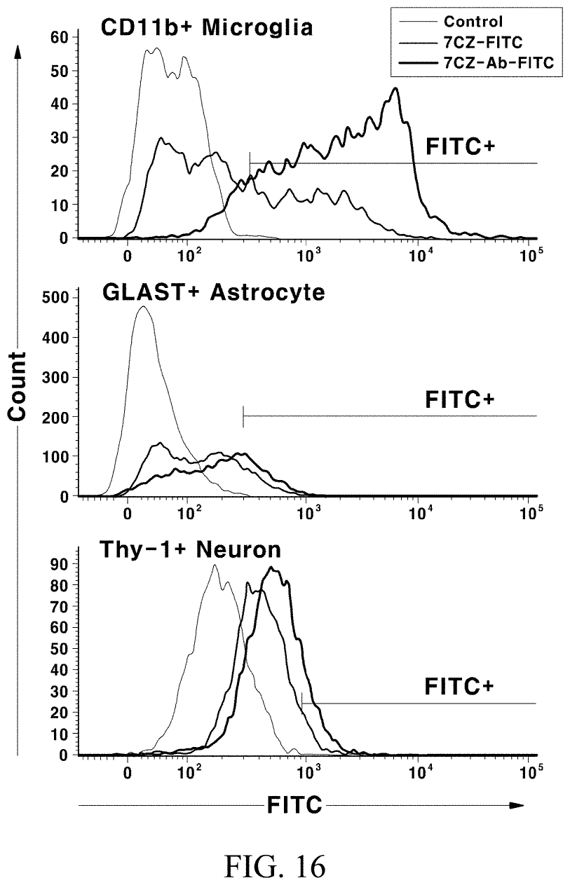

FIGS. 12 to 16 are views for explaining microglia specific delivery of nanoparticle structures according to an embodiment of the present invention.

Referring to FIGS. 12 to 16, CD11b-Ab bonding induces increased absorption of 7CZ nanoparticles by microglia and imparts specificity to microglia in vivo. In order to confirm the uptake of 7CZ-Ab nanoparticles in vivo, the nanoparticles were injected into the spinal cord of a mouse and analyzed through IHC (immunohistochemistry) 24 hours later.

L4-L6 tissue samples are immunostained with cell type specific antibodies, and localization of the FITC signal is observed. FITC signals are localized primarily in Iba-1+ microglia and not localized in GFAP+ astrocytes and MAP-2+ neurons. FITC-positive cells were analyzed in the spinal cord by flow cytometry 24 hours after 7CZ-Ab-FITC administration.

In characterization of 7CZ Ab-FITC positive cells using cell type specific markers, FITC signals are observed in 84% of CD11b+ microglia, 26% of GLAST+ astrocytes, and 11% of Thy-1+ neurons. Compared to astrocytes and neurons, a significantly higher percentage of microglia cells showed uptake of 7CZ-Ab nanoparticles.

The MFI of microglia cells is much higher than that of astrocytes and neurons. Administration of 7CZ-Ab nanoparticles to mice showed a significant difference in the absorption characteristics of CD11b+ microglia and the specificity of nanoparticles to microglia as a result of CD11b-Ab bonding to 7CZ nanoparticles.

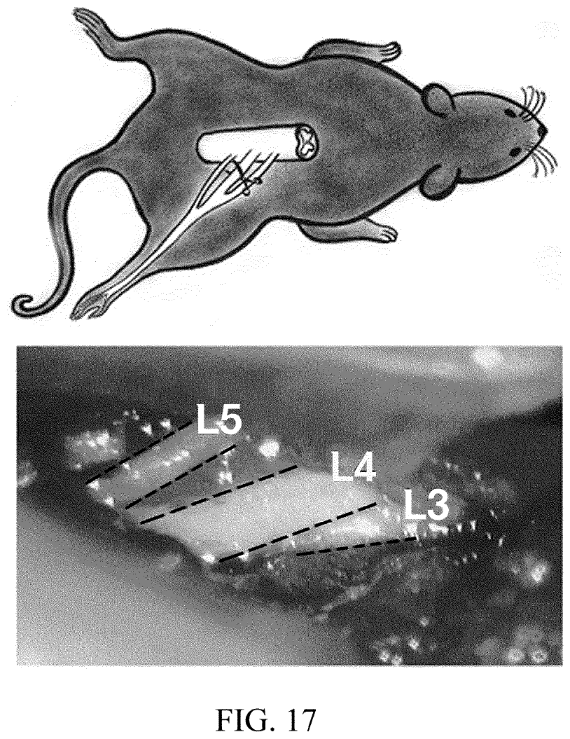

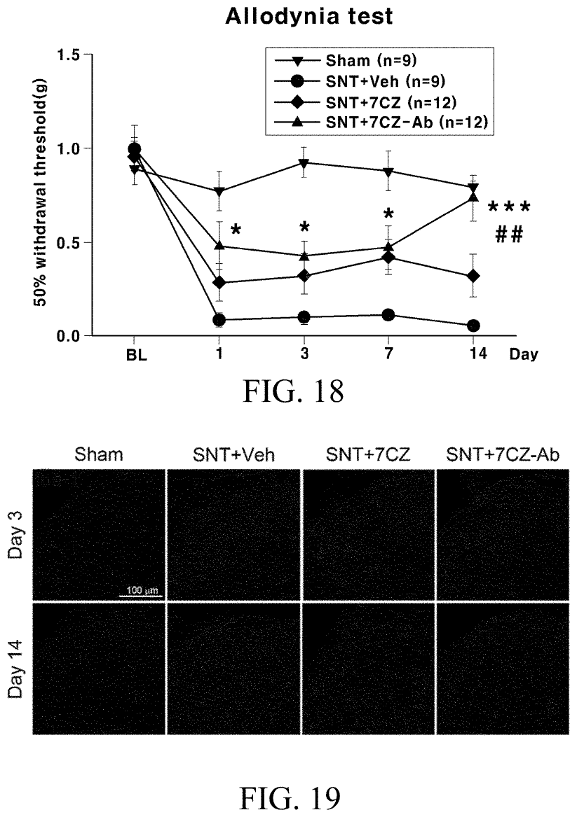

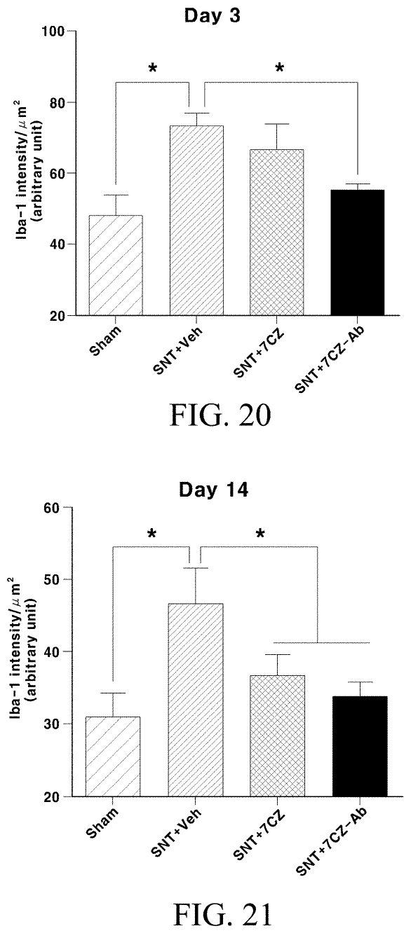

FIGS. 17 to 21 are views for explaining an analgesic effect of nanoparticle structures according to an embodiment of the present invention.

Referring to FIGS. 17 to 21, in order to analyze whether microglia targeting by 7CZ-Ab nanoparticles has a better analgesic effect, the susceptibility to nerve damage-induced pain hypersensitivity in mice treated with 7CZ nanoparticles or 7CZ-Ab nanoparticles was compared. After L5 SNT, SNT mice showed increased sensitivity to mechanical stimulation as measured by the Vonfrey test. PWT for mechanical stimulation decreased from about 0.99 g to less than 0.1 g 1 day after injury.

The threshold was kept below 0.1 g for 2 weeks. PWT was measured for mice pretreated with the same molar concentration of 7CZ nanoparticles or 7CZ-Ab nanoparticles administered through the spinal canal route. In mice treated with 7CZ nanoparticles, PWT increased to 0.28 g at ldpi, 0.32 g at 3 dpi, 0.42 g at 7 dpi, and 0.32 g at 14 dpi, indicating that mechanical allodynia was moderately reduced. In mice treated with 7CZ-Ab nanoparticles, PWT increased to 0.48 g at ldpi, 0.42 g at 3 dpi, 0.47 g at 7 dpi, and 0.73 g at 14 dpi, indicating that 7CZ-Ab nanoparticles have a stronger analgesic effect compared to 7CZ nanoparticles.

A significant increase in the withdrawal threshold (WT) due to treatment with 7CZ-Ab nanoparticles compared to 7CZ nanoparticles (14 days, p value=0.023) means a greater therapeutic effect for neuropathic pain treatment. This may be due to the increased micrglia targeting efficiency.

To evaluate the effect of 7CZ nanoparticles and 7CZ-Ab nanoparticles on spinal cord microglia activity in neuropathic pain induced by nerve injury in vivo, nanoparticles were administered prior to L5 SNT. Sham mice and mice with SNT damage were injected with 7CZ nanoparticles or 7CZ-Ab nanoparticles, and after 3 and 14 days, Iba-1 (microglia cell marker) intensity was measured and compared in spinal cord sections of mice.

In the spinal cord dorsal horns (ipsilateral) of SNT-injured mice, Iba-1 fluorescence intensity increased by 0.54 times for 3 days and 0.45 times for 14 days compared to that of the sham mice. However, SNT-induced microglia activation decreased by 30% in mice treated with 7CZ-Ab nanoparticles for days, and decreased by 19% in mice treated with 7CZ nanoparticles.

At the time of 14 days, the activation of SNT-induced microglia cells decreased by 22% by administration of 7CZ-Ab nanoparticles and decreased by 20% by administration of 7CZ nanoparticles. Although both types of nanoparticles mitigate SNT-induced microglia activation, 7CZ-Ab nanoparticles initially showed a greater reduction in microglia activation compared to 7CZ nanoparticles. In vivo, 7CZ-Ab nanoparticles more effectively inhibit SNT-induced spinal cord microglia activity than 7CZ nanoparticles.

As described above, targeting the nanoparticles to the microglia cells may result in more absorption of the nanoparticles by the microglia, thereby enhancing a greater reactive oxygen species scavenging effect and a reduction in pro-inflammatory gene expression. In other words, the higher microglial uptake of antibody-bonded 7CZ (7CZ-Ab) nanoparticles than 7CZ nanoparticles can further reduce microglia activation by reducing more reactive oxygen species and pro-inflammatory cytokines in vitro and in vivo. In addition, CD11b bonding to 7CZ nanoparticles can impart specificity to microglia in vivo. These nanoparticles can reduce microglia activation and suppress mechanical allodynia with a single dose of intrathecal injection. Therefore, CD11b-bonded 7CZ nanoparticles can be applied as an analgesic for the treatment of neuropathic pain.

The automatic conversion of the two oxidation states (Ce³⁺ and Ce⁴⁺) through electrons in ceria nanoparticles allows the particles to continuously scavenge reactive oxygen species. In addition, since Ce³⁺ ions are more important than Ce⁴⁺ ions to improve inflammatory diseases, it is necessary to insert other dopant ions to maintain high Ce³⁺ content of ceria nanoparticles.

Ceria-zirconia nanoparticles show greater activity in the elimination of harmful molecules, superoxide anion (O₂⁻) and hydroxyl radical (.OH) in the pathogenesis of inflammatory diseases, thereby enabling improved treatment in various disease models. In addition, targeted delivery approaches for nanotherapies can enhance the efficacy of drugs while avoiding side effects, and targeting and treating diseased areas can provide a higher recovery rate for patients.

For example, to relieve the activation of microglia which is a major cause of pain, ceria-zirconia nanoparticles (Ce_{0.7}Zr_{0.3}O₂) with a size of 2 nm show good reactive oxygen species scavenging performance. In addition, by functionalizing the microglia targeting antibody (CD11b) on the nanoparticles, it is possible to block the generation of reactive oxygen species and inflammatory reactions in the early stages of microglia activation.

Although the embodiments of the present invention have been disclosed for illustrative purposes, those skilled in the art will appreciate that the present invention may be embodied in other specific ways without changing the technical spirit or essential features thereof. Therefore, the embodiments disclosed in the present invention are not restrictive but are illustrative. The scope of the present invention is given by the claims, rather than the specification, and also contains all modifications within the meaning and range equivalent to the claims.

### INDUSTRIAL APPLICABILITY

According to embodiments of the present invention, a novel nanoparticle structure having a good therapeutic effect may be implemented. For example, the nanoparticle structure may inhibit microglia activation. The targeted delivery of the nanoparticle structure induces improved elimination of inflammatory cytokines and reactive oxygen species in microglia to enable down-regulation of hyperactivated microglia. Thereby, neuropathic pain induced by microglia activation can be suppressed. In addition, the nanoparticle structure may be used for the treatment of various diseases related to microglia activation and may be applied to other targeted treatments other than microglia.

1. A nanoparticle structure comprising a metal oxide nanoparticle.

2. The nanoparticle structure of claim 1, wherein the metal oxide nanoparticle comprises ceria.

3. The nanoparticle structure of claim 2, wherein the metal oxide nanoparticle further comprises zirconia.

4. The nanoparticle structure of claim 3, wherein the metal oxide nanoparticle has a formula of Ce_{0.7}Zr_{0.3}O₂.

5. The nanoparticle structure of claim 1, further comprising an antibody connected to the metal oxide nanoparticle.

6. The nanoparticle structure of claim 4, wherein the antibody comprises a microglia targeting antibody.

7. The nanoparticle structure of claim 6, wherein the nanoparticle structure relieves neuropathic pain.

8. The nanoparticle structure of claim 6, wherein the nanoparticle structure inhibits microglia activation.

9. A method of forming a nanoparticle structure comprising forming a metal oxide nanoparticle.

10. The method of claim 9, wherein the metal oxide nanoparticle is formed by putting cerium(III) acetylacetonate hydrate and zirconium(IV) acetylacetonate hydrate in oleylamine and heating the cerium(III) acetylacetonate hydrate and the zirconium(IV) acetylacetonate hydrate to react.

11. The method of claim 9, further comprising connecting an antibody to the metal oxide nanoparticle.

12. The method of claim 11, wherein connecting the antibody to the metal oxide nanoparticle comprises connecting phospholipid-PEG to the metal oxide nanoparticle and reacting the antibody with the phospholipid-PEG.

13. The method of claim 12, wherein the antibody reacts with NHS-ester before the antibody is connected to the metal oxide nanoparticle and reacts with the phospholipid-PEG to be connected to the metal oxide nanoparticle by amide bond.
